(19) United States
(12) Patent Application Publication
(10) Pub. No.: US 2013/0226029 A1
(43) Pub. Date: Aug. 29, 2013

(54) TWO-PART ACCESS PORT

(71) Applicant: COVIDIEN LP, (US)

(72) Inventor: Gennady Kleyman, Brooklyn, NY (US)

(73) Assignee: COVIDIEN LP, Mansfield, MA (US)

(21) Appl. No.: 13/746,520

(22) Filed: Jan. 22, 2013

Related U.S. Application Data
(60) Provisional application No. 61/602,102, filed on Feb. 23, 2012.

(51) Int. Cl. A61B 10/02; A61B 17/02

(52) U.S. Cl. CPC A61B 10/0266; A61B 17/0218
USPC 600/564; 600/208

(57) ABSTRACT

A surgical access apparatus has a body including a proximal portion, a distal portion, and a liner interconnecting the proximal portion and the distal portion. The proximal portion and the distal portion are longitudinally movable relative to each other. A holder is releasably attached to the body for holding the body in a first state, in which the proximal portion and the distal portion are a first distance apart. Removing the holder from the body allows the body to transition to a second state, in which the proximal portion and the distal portion are a second distance apart. The second distance is greater than the first distance. At least one port extends through the proximal portion and is adapted for the reception of an object. Compressible material defining the at least one port is adapted to deform to establish a substantial sealed relation with the object.

### CROSS REFERENCE TO RELATED APPLICATION

The present application claims the benefit of and priority to U.S. Provisional Application Ser. No. 61/602,102, filed on Feb. 23, 2012, the entire contents of which are incorporated herein by reference.

### BACKGROUND

1. Technical Field

The present disclosure relates to a surgical apparatus for use during a minimally invasive surgical procedure. More particularly, the present disclosure relates to a surgical access apparatus adapted for insertion into an opening in tissue, and, for the sealed reception of one or more surgical objects such that a substantially fluid-tight seal is formed with both the tissue and the surgical object, or objects.

2. Background of the Related Art

Minimally invasive surgery, e.g., laparoscopic, endoscopic, and thoroscopic surgery, has many advantages over traditional open surgeries. In particular, minimally invasive surgery eliminates the need for a large incision, thereby reducing discomfort, recovery time, and many of the deleterious side effects associated with traditional open surgery.

Minimally invasive surgeries are performed through small openings in a patient's skin. These openings may be incisions in the skin or may be naturally occurring body orifices (e.g., mouth, anus, or vagina). During a typical minimally invasive procedure, surgical objects, such as surgical access devices, e.g., trocar and cannula assemblies, or endoscopes, are inserted into the patient's body through the incision in tissue. In general, insufflation fluid (e.g., CO₂ or saline) is used to enlarge the area surrounding the target surgical site to create a larger, more accessible work area. Accordingly, the maintenance of a substantially fluid-tight seal is desirable so as to minimize the escape of the insufflation fluids and the deflation or collapse of the enlarged surgical site. To this end, various valves and seals are used during the course of minimally invasive procedures and are widely known in the art.

Removal of a specimen from a surgical site is desirable during many minimally invasive procedures. The specimen may be wider than an access opening of a valve or seal, thus requiring a removal of the valve or seal before the specimen can be removed, which may result in increased duration, complexity, and/or cost of the procedure. A continuing need exists for a surgical access apparatus that can be used both for maintaining the integrity of an insufflated workspace and for facilitating a removal of a specimen from the workspace.

### SUMMARY

A surgical access apparatus has a body including a proximal portion, a distal portion, and a liner interconnecting the proximal portion and the distal portion. The proximal portion and the distal portion are longitudinally movable relative to each other. A holder is releasably attached to the body for holding the body in a first state, in which the proximal portion and the distal portion are a first distance apart. Removing the holder from the body allows the body to transition to a second state, in which the proximal portion and the distal portion are a second distance apart. The second distance is greater than the first distance. At least one port extends through the proximal portion and is adapted for the reception of an object. Compressible material defining the at least one port is adapted to deform to establish a substantial sealed relation with the object.

The holder may be a suture. The holder may be welded to the body. The distal portion may have an aperture substantially wider than each of the at least one port. The liner may be collapsed in the first state and expanded in the second state. The distal portion may include a bag portion for carrying a specimen. The compressible material may be a foam or a plastic.

A method of retrieving a specimen has the steps of inserting a surgical access apparatus into an opening, releasing a holder from the body of the surgical access apparatus, moving a proximal portion and a distal portion of the body longitudinally apart, moving a specimen within the liner of the body, and removing the body from the opening. The liner interconnects the proximal portion and the distal portion. The body further defines at least one port configured for forming a substantial sealed relation with a surgical instrument inserted through the at least one port.

The releasing step may include cutting the holder. The releasing step may include removing the holder from the body. The surgical access apparatus may include a bag for carrying a specimen. The step of moving a proximal portion and a distal portion may include stretching the liner. The step of moving a specimen within the liner may be performed by a surgical instrument inserted through the at least one port.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a side, perspective view of a surgical access apparatus according to an embodiment of the present disclosure;

FIG. 2 is a side, cross-section view of the surgical access apparatus of FIG. 1 in a first configuration;

FIG. 3 is a side, cross-section view of the surgical access apparatus of FIG. 1 with holders removed in a second configuration;

FIG. 4 is a side, cross-section view of the surgical access apparatus of FIG. 1 in a first condition prior to insertion thereof within an opening in a tissue;

FIG. 5 is a side, cross-section view of the surgical access apparatus of FIG. 1 inserted through an opening in tissue and having a holder cut;

FIG. 6 is a side, cross-section view of the surgical access apparatus of FIG. 1 in a second configuration in an opening in tissue and having a surgical instrument inserted therethrough;

FIG. 7 is a side, cross-section view of the surgical access apparatus of FIG. 1 having a specimen placed within a liner thereof; and

FIG. 8 is a side, cross-section view of the surgical access apparatus of FIG. 1 removed from an opening in a tissue and having a specimen therein.

### DETAILED DESCRIPTION

Particular embodiments of the present disclosure are described hereinbelow with reference to the accompanying drawings; however, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Like reference numerals may refer to similar or identical elements throughout the description of the figures. As shown in the drawings and described throughout the following description, as is traditional when referring to relative positioning on a surgical instrument, the term “proximal” refers to the end of the apparatus which is closer to the user and the term “distal” refers to the end of the apparatus which is farther away from the user. The term “clinician” refers to any medical professional (i.e., doctor, surgeon, nurse, or the like) performing a medical procedure involving the use of embodiments described herein.

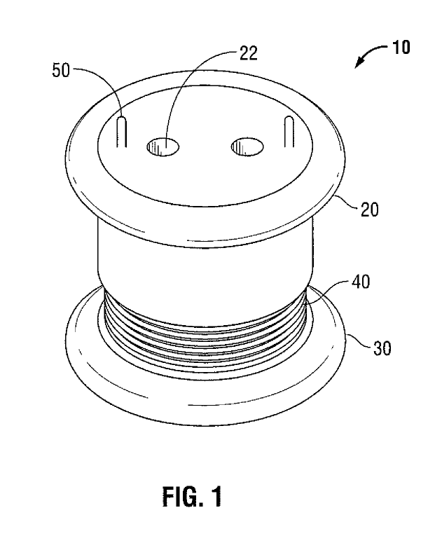

FIG. 1 illustrates a surgical access apparatus 10 for use in a surgical procedure, e.g., a minimally invasive procedure. Surgical access apparatus 10 has a body including a proximal portion 20, a distal portion 30, and a liner 40 interconnecting proximal portion 20 and distal portion 30. Proximal portion 20 includes at least one port 22 extending longitudinally therethrough. At least one port 22 is configured for removably receiving a surgical object therethrough.

Surgical access apparatus 10 may be composed of any suitable combination of biocompatible materials, such as biocompatible metals, plastics, and foams. In one embodiment, surgical access apparatus 10 includes a foam sufficiently compliant to accommodate off-axis motion of a surgical object. The foam may be a polyisoprene material. Proximal portion 20 may be composed of a material having sufficient compliance to form a substantially fluid-tight seal about one or more surgical objects within at least one port 22. At least one port 22 has an initial diameter without an object inserted therethrough. The initial diameter may be substantially zero mm. Insertion of a surgical object through at least port 22 expands at least port 22 from the initial diameter to at least a diameter of the surgical object. Resilient material defining at least one port 22 attempts to resiliently restore at least one port 22 to the initial diameter, thereby forming a substantially fluid-tight seal about the surgical object. Proximal portion 20 and distal portion 30 may be composed of a resiliently compressible material to allow surgical access apparatus 10 to resiliently transition between a compressed condition to facilitate insertion of surgical apparatus 10 and an expanded condition to substantially secure surgical access apparatus 10 within a bodily opening. After being compressed from an uncompressed state for insertion, the resiliently compressible material defining proximal portion 20 and distal portion 30 attempts to resiliently expand to the uncompressed state, thereby forming a substantially fluid-tight seal with tissue defining a bodily opening.

Proximal portion 20 may have a proximal end sufficiently wide to inhibit surgical access apparatus 10 from passing completely through a bodily opening during a surgical procedure. Distal portion 30 may likewise have a distal end sufficiently wide to inhibit unintended removal of surgical access apparatus 10 from a bodily opening during a surgical procedure. The proximal end and the distal end may be planar. In another embodiment, the proximal end and the distal end may be substantially arcuate to assist in the insertion of surgical apparatus 10 within a bodily opening. Proximal end 20 may have a body portion distal to the proximal end thereof having a diameter appreciably less than a diameter of the proximal end. Distal portion 30 may likewise have a body portion proximal to the distal end thereof having a diameter appreciably less than a diameter of the distal end. Surgical apparatus 10 may define an “hour-glass” shape or configuration to assist in anchoring surgical apparatus 10 within tissue. In alternate embodiments, proximal end 20 and distal end 30 may have a substantially uniform diameter. The body portions of proximal portion 20 and distal portion 30 may exhibit any suitable configuration, e.g., substantially circular, oval, or oblong.

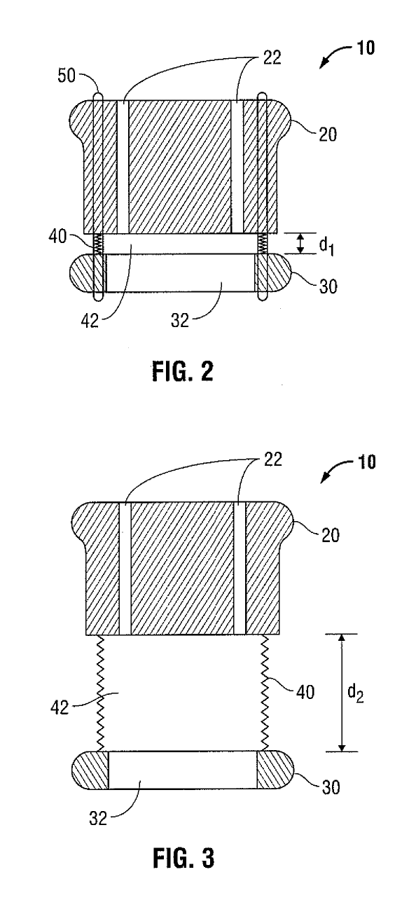

As seen in FIG. 2, surgical access apparatus 10 includes a holder 50 for holding the body of surgical access apparatus 10 in a first state. In the first state, proximal portion 20 and distal portion 30 are a first distance d₁ longitudinally apart. The first distance d₁ is preferably minimal. Proximal portion 20 and distal potion 30 may be in contact. Liner 40 is compressed, folded, crumpled, or otherwise shortened along a longitudinal dimension of surgical access apparatus 10 to assist in minimizing the first distance d₁.

Holder 50 inhibits proximal portion 20 and distal portion 30 from moving longitudinally apart beyond the first distance d₁. Surgical access apparatus 10 may include more than one holder 50. Holder 50 extends from a proximal end of proximal portion 20 through the body to distal portion 30. In one embodiment, holder 50 is a suture. In another embodiment, holder 50 is welded to the body. A portion of holder 50 on the proximal end of proximal portion 20 may be cut, pulled, disengaged, or otherwise released from the body to allow the body to transition to a second state.

Distal portion 30 has an aperture 32 defined therein. Aperture 32 is sufficiently wide for a specimen to pass therethrough. Aperture 32 also allows passage of surgical instruments therethrough. Aperture 32 may be wider than each of at least one port 22.

Liner 40 is sufficiently longitudinally expandable for a space 42 therein to be sufficiently large to receive a specimen therein. Liner 40 may longitudinally extend through an entire depth of an opening in a fully expanded state. Space 42 may be wider than aperture 32, thus allowing distal portion 30 to assist in carrying a specimen within space 42. In some Embodiments, liner 40 may include a net, bag, or other carrying device for carrying a specimen therein. Liner 40 may be composed of a relatively thin, flexible material adapted for compression and expansion. Liner 40 is substantially impermeable to reduce seeding or spreading of the specimen.

As seen in FIG. 3, the body of surgical access apparatus 10 is in a second state. Holder 50 is removed from the body of surgical access apparatus 10 and no longer inhibits longitudinal movement of proximal portion 20 relative to distal portion 30. Liner 40 is longitudinally expanded, thereby increasing a volume of space 42. Proximal end 20 and distal end 30 are at a second distance d₂ from each other greater than the first distance d₁.

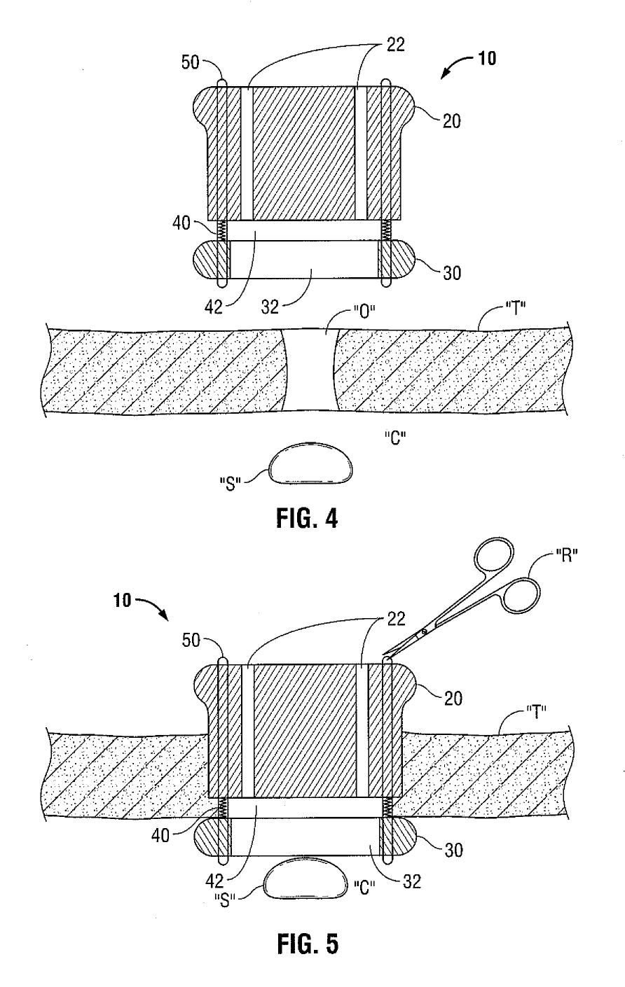

Turning to FIGS. 4-8, a method of use of surgical access apparatus 10 is illustrated. As seen in FIG. 4, an opening “0” exists in a tissue “T” through which a cavity “C” may be accessed. A specimen “S” exists in cavity “C”. Surgical access apparatus 10 is compressible to a diameter less than a diameter of opening “O” to facilitate insertion of surgical access apparatus 10 in opening “O”. The body of surgical access apparatus 10 is in the first state when inserted. When surgical access apparatus 10 is within opening “O”, surgical access apparatus 10 resiliently expands until it forms a substantial sealed relation with tissue “T”. Surgical instruments may then be inserted through at least one port 22 and continue through space 42 and aperture 32 to access cavity “C”.

As seen in FIG. 5, once specimen “S” is ready for removal, holder 50 may be cut by a cutting instrument “R” to release holder 50 from the body of surgical access apparatus 10. Holder 50 may then be pulled proximally for removal thereof from the body.

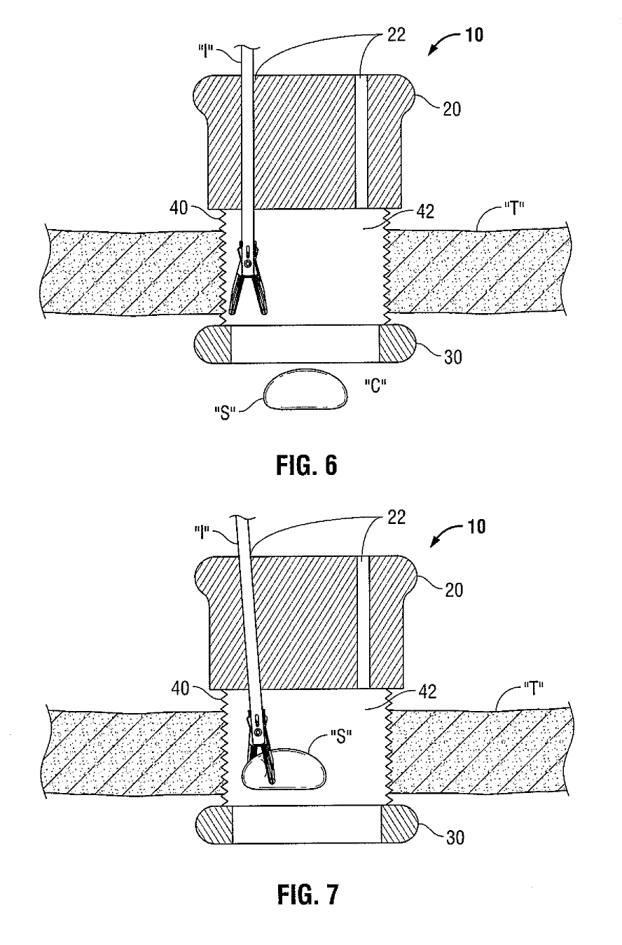

As seen in FIG. 6, the body of surgical access apparatus 10 is transitioned to the second state. Distal portion 30 is positioned distal to tissue “T”. Proximal portion 20 is pulled proximally until it is positioned fully proximal to tissue “T”. Liner 40 expands longitudinally along an entire depth of opening “O”. A grasping instrument “I” is then inserted through at least one port 22. Compressible material defining at least one port 22 forms a substantially fluid-tight seal around grasping instrument “I”.

As seen in FIG. 7, grasping instrument “I” grasps specimen “S” and pulls specimen “S” through aperture 32 into space 42. Grasping instrument “I” may continue to grasp specimen “S” during removal. In embodiments having a carrying device such as a bag, specimen “S” may be placed within the carrying device by grasping instrument “I”. Alternatively, a carrying device could be provided by a surgical instrument inserted through at least one port 22.

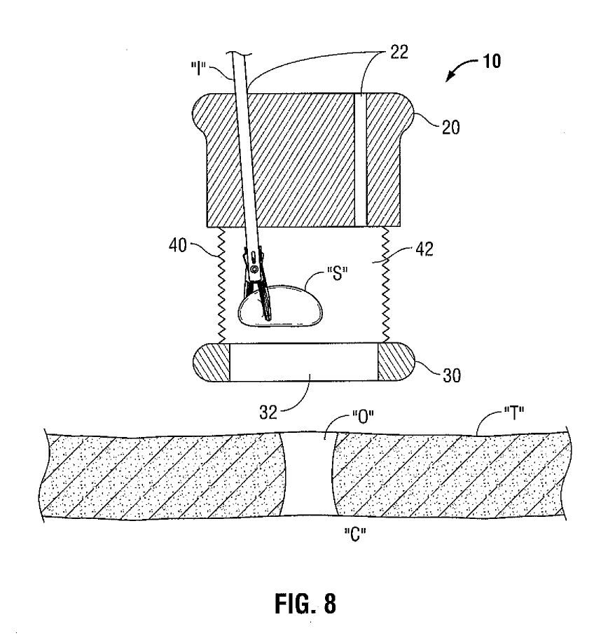

As seen in FIG. 8, surgical access apparatus 10 is removed from opening “O” to complete the removal method. Distal portion 30 is compressed to a diameter less than a diameter of opening “O”. Surgical access apparatus 10 may then be pulled proximally from tissue “T”. If no carrying device is provided, grasping instrument “I” is pulled proximally with surgical access apparatus 10 to keep specimen “S” within space 42 as surgical access apparatus 10 is removed from opening “O”. Distal portion 30 resiliently expands upon removal from opening “O”.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figs. are presented only to demonstrate certain examples of the disclosure. Other elements, steps, methods and techniques that are insubstantially different from those described above and/or in the appended claims are also intended to be within the scope of the disclosure.

What is claimed is:

1. A surgical access apparatus comprising:
a body including a proximal portion, a distal portion, and a liner interconnecting the proximal portion and the distal portion, the proximal portion and the distal portion being longitudinally movable relative to each other;
a holder releasably attached to the body for holding the body in a first state, wherein the proximal portion and the distal portion are a first distance apart, wherein releasing the holder from the body allows the body to transition to a second state, wherein the proximal portion and the distal portion are a second distance apart, the second distance being greater than the first distance; and
at least one port extending through the proximal portion and being adapted for the reception of an object whereby compressible material defining the at least one port is adapted to deform to establish a substantial sealed relation with the object.

2. The surgical access apparatus of claim 1, wherein the holder is a suture.

3. The surgical access apparatus of claim 1, wherein the holder is welded to the body.

4. The surgical access apparatus of claim 1, wherein the distal portion has an aperture substantially wider than each of the at least one port.

5. The surgical access apparatus of claim 1, wherein the liner is collapsed in the first state and expanded in the second state.

6. The surgical access apparatus of claim 1, wherein the distal portion includes a carrying device for carrying a specimen therein.

7. The surgical access apparatus of claim 1, wherein the compressible material is a foam.

8. The surgical access apparatus of claim 1, wherein the compressible material is a plastic.

9. A method of retrieving a specimen, comprising the steps of:
inserting a surgical access apparatus into an opening, wherein the surgical access apparatus has a holder and a body, the body including a liner interconnecting a proximal portion and a distal portion of the body, the body further defining at least one port configured for forming a substantial sealed relation with a surgical instrument inserted therethrough;
releasing the holder from the body;
moving the proximal portion and the distal portion longitudinally apart;
moving a specimen within the liner; and
removing the body from the opening.

10. The method of claim 9, wherein the releasing step includes cutting the holder.

11. The method of claim 9, wherein the releasing step includes removing the holder from the body.

12. The method of claim 9, wherein the surgical access apparatus includes a carrying device for carrying a specimen.

13. The method of claim 9, wherein the step of moving the proximal portion and the distal portion longitudinally apart includes stretching the liner.

14. The method of claim 9, wherein the step of moving a specimen within the liner is performed by a surgical instrument inserted through the at least one port.
